(19) United States
(12) Patent Application Publication
(10) Pub. No.: US 2026/0188491 A1
(43) Pub. Date: Jul. 2, 2026

(54) INTELLIGENT MEDICAL CONSULTATION DIALOGUE METHOD, APPARATUS, COMPUTER DEVICE, AND STORAGE MEDIUM

(71) Applicant: Beijing Yilian Zhishu Technology Co., Ltd., Beijing (CN)

(72) Inventor: Shirui WANG, Beijing (CN)

(73) Assignee: Beijing Yilian Zhishu Technology Co., Ltd., Beijing (CN)

(21) Appl. No.: 19/391,485

(22) Filed: Nov. 17, 2025

(30) Foreign Application Priority Data
Dec. 30, 2024 (CN) 202411976041.4

(51) Int. Cl. G16H 50/20; G16H 40/67

(52) U.S. Cl. CPC G16H 50/20; G16H 40/67

(57) ABSTRACT

The present disclosure provides an intelligent medical consultation dialogue method, including: acquiring a medical consultation dialogue from a target user through an intelligent medical consultation dialogue interface; extracting disease feature information from the consultation dialogue; querying a medical decision-making database based on the disease feature information to obtain a suspected disease set; calculating a degree of suspicion corresponding to the suspected disease in the suspected disease set; screening for a target suspected disease from the suspected disease set based on the degree of suspicion; determining whether an unconfirmed disease factor exists in the suspected disease based on the disease feature information; and initiating a question generation task based on the unconfirmed disease factor, performing the question generation task using a large language model to obtain a question targeting the unconfirmed disease factor, and outputting the question. The method provides a transparent and controllable consultation process, reducing risk of diagnostic errors.

### CROSS-REFERENCE TO RELATED APPLICATIONS

This application claims priority of Chinese Patent Applicant No. 202411976041.4, filed on Dec. 30, 2024, entitled as “Intelligent Medical Consultation Dialogue Method, Apparatus, Computer Device, and Storage Medium,” the entire disclosure of which is incorporated herein by reference for all purposes.

### TECHNICAL FIELD

The present disclosure relates to the field of computer technology, and more particularly to an intelligent medical consultation dialogue method, an apparatus, a computer device, and a storage medium.

### BACKGROUND

With the development of artificial intelligence technology, an increasing number of artificial intelligence medical consultation systems are being applied to clinical auxiliary diagnosis and health management.

However, existing artificial intelligence medical consultation systems still have certain shortcomings, especially in terms of the logic and controllability of the consultation process. These systems typically rely on black-box models for symptom matching and disease inference, lacking a transparent reasoning process and deterministic results, which makes the diagnostic process difficult to understand and supervise. Furthermore, in the analysis of complex cases or multiple symptoms, current systems often fail to provide sufficient explanations and reasonable decision support, thereby affecting the final diagnostic accuracy and user experience.

Therefore, designing an intelligent consultation method with controllable diagnostic logic has become an urgent need that must be addressed.

### SUMMARY

Embodiments of the present disclosure provide an intelligent medical consultation dialogue method, an apparatus, a computer device, and a storage medium, which can enhance the rationality of the intelligent consultation process and the accuracy of diagnostic results.

In a first aspect, an embodiment of the present disclosure provides an intelligent medical consultation dialogue method, including:
acquiring a medical consultation dialogue from a target user through an intelligent medical consultation dialogue interface;
extracting disease feature information from the consultation dialogue, where the disease feature information includes one or more of symptom information, sign information, and risk factor information;
querying a medical decision-making database based on the disease feature information to obtain a suspected disease set, where the suspected disease set contains one or more suspected diseases;
calculating a degree of suspicion corresponding to the suspected disease in the suspected disease set;
screening for a target suspected disease from the suspected disease set based on the degree of suspicion;
determining whether an unconfirmed disease factor exists in the suspected disease based on the disease feature information; and
in response to determining that one or more unconfirmed disease factors exist in the target suspected disease, initiating a question generation task based on the unconfirmed disease factor, performing the question generation task using a large language model to obtain a question targeting the unconfirmed disease factor, and outputting the question through the intelligent medical consultation dialogue interface.

In a second aspect, an embodiment of the present disclosure provides an intelligent medical consultation dialogue apparatus, including:
an acquirer, configured to acquire a medical consultation dialogue from a target user through an intelligent medical consultation dialogue interface;
a feature extractor, configured to extract disease feature information from the consultation dialogue, where the disease feature information includes one or more of symptom information, sign information, and risk factor information;
a querier, configured to query a medical decision-making database based on the disease feature information to obtain a suspected disease set, where the suspected disease set contains one or more suspected diseases;
a calculator, configured to calculate a degree of suspicion corresponding to the suspected disease in the suspected disease set;
a screener, configured to screen for a target suspected disease from the suspected disease set based on the degree of suspicion;
an analyzer, configured to determine whether an unconfirmed disease factor exists in the target suspected disease based on the disease feature information; and
a dialoguer, configured to, in response to determining that one or more unconfirmed disease factors exist in target the suspected disease, initiate a question generation task based on the unconfirmed disease factor, perform the question generation task using a large language model to obtain a question targeting the unconfirmed disease factor, and output the question through the intelligent medical consultation dialogue interface.

In a third aspect, an embodiment of the present disclosure provides a computer device, including a processor, and a memory storing computer programs, where the processor is configured to read and execute the computer programs to implement the method according to the first aspect mentioned above.

In a fourth aspect, an embodiment of the present disclosure provides a non-transitory computer-readable storage medium, having computer programs or instructions stored thereon, where the computer programs or instructions, when executed by a processor, implement the method according to the first aspect mentioned above.

### BRIEF DESCRIPTION OF DRAWINGS

The drawings herein are incorporated into the specification and constitute a part of this specification, illustrating embodiments that conform to the present disclosure, and are used together with the specification to explain the principles of the present disclosure, and do not constitute an improper limitation of the present disclosure.

FIG. 1 is a schematic flowchart of an intelligent medical consultation dialogue method according to an embodiment of the present disclosure;

FIG. 2 is a schematic flowchart of an intelligent medical consultation dialogue method according to an embodiment of the present disclosure;

FIG. 3 is a schematic diagram showing model structure of a disease analysis model according to an embodiment of the present disclosure;

FIG. 4 is a schematic flowchart of a training method for a disease analysis model according to an embodiment of the present disclosure;

FIG. 5 is a schematic flowchart of a prediction method for a disease analysis model according to an embodiment of the present disclosure;

FIG. 6 is a schematic flowchart of an intelligent medical consultation dialogue method according to an embodiment of the present disclosure;

FIG. 7 is a schematic structural diagram of an intelligent medical consultation dialogue apparatus according to an embodiment of the present disclosure; and

FIG. 8 is a schematic structural diagram of a computer device according to an embodiment of the present disclosure.

### DETAILED DESCRIPTION OF THE EMBODIMENTS

To enable a person of ordinary skill in the art to better understand the technical solutions of the present disclosure, the technical solutions in the embodiments of the present disclosure will be described clearly and completely below with reference to the accompanying drawings. It should be understood that the specific embodiments described herein are only intended to explain the present disclosure, not to limit it. For those skilled in the art, the present disclosure can be implemented without some of these specific details. The following description of the embodiments is merely to provide a better understanding of the present disclosure by showing examples of the present disclosure.

It should be noted that terms such as “first” and “second” in the specification and claims of the present disclosure and the aforementioned drawings are used to distinguish similar objects and not necessarily to describe a specific order or sequence. It should be understood that the data so used can be interchanged under appropriate circumstances, so that the embodiments of the present disclosure described herein can be implemented in an order other than those illustrated or described here. The implementations described in the following exemplary embodiments do not represent all implementations consistent with the present disclosure. Instead, they are merely examples consistent with some aspects of the present disclosure, as detailed in the appended claims.

As described in the background, there is currently no good solution in the related art to ensure sufficient controllability and transparency in the consultation process of artificial intelligence medical consultation systems. To solve this technical problem, embodiments of the present disclosure provide an intelligent medical consultation dialogue method, an apparatus, a computer device, and a storage medium, which utilize a large language model to conduct consultation dialogues with users, accurately understand the semantics of user input and generate relevant consultation questions, guiding users to provide more detailed and accurate disease feature information; moreover, with the assistance of a medical decision-making system, medical decision-making tasks are executed based on the acquired disease feature information to generate reasonable diagnostic inferences and suggestions.

The intelligent medical consultation dialogue method provided by the embodiments of the present disclosure will be described in detail below with reference to the accompanying drawings, through specific embodiments and their application scenarios.

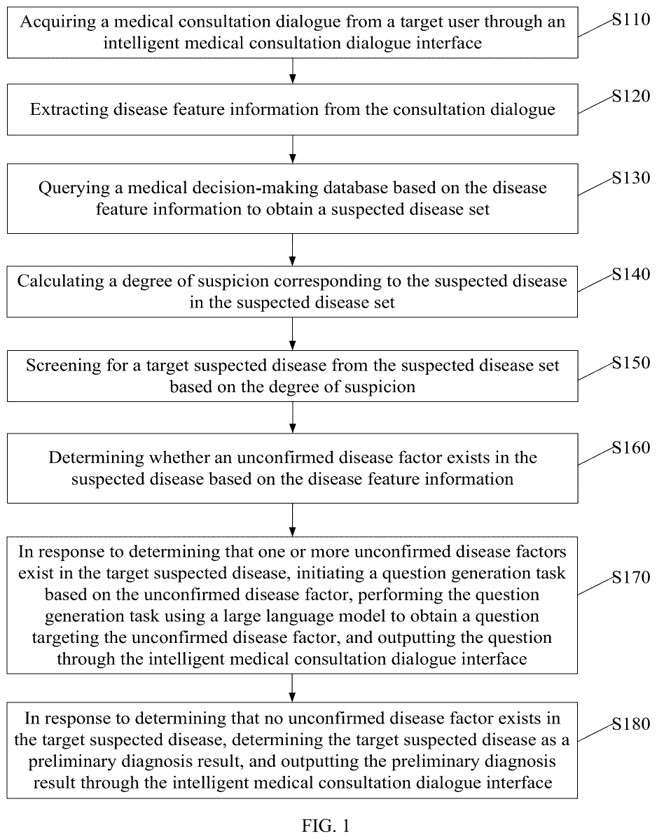

FIG. 1 is a schematic flowchart of an intelligent medical consultation dialogue method according to an embodiment of the present disclosure.

As shown in FIG. 1, the method may include the following steps.

S110, acquiring a medical consultation dialogue from a target user through an intelligent medical consultation dialogue interface.

It can be understood that the application subject of the method of the present disclosure may be an intelligent medical consultation system or an intelligent medical consultation platform.

Based on the intelligent medical consultation dialogue method provided by the embodiments of the present disclosure, the system or platform may interact with the user, collect information provided by the user, and combine it with medical decision-making algorithms to generate appropriate consultation questions, and perform disease analysis and reasoning based on the user's answers.

The intelligent medical consultation dialogue interface refers to the channel for interaction between the intelligent medical consultation system and the user.

As an example, the intelligent medical consultation dialogue interface may be set up in the form of an application, a web interface, or a chatbot.

The target user refers to the individual using the intelligent medical consultation system for a consultation.

As an example, the target user may be a patient, a health management professional, a doctor, or anyone with a need for medical consultation.

The medical consultation dialogue refers to the dialogue information between the target user and the intelligent medical consultation system, containing the target user's health status and related medical information.

As an example, the medical consultation dialogue may include, but is not limited to, the following types of information:

User's basic information: such as gender, age, height, weight, etc.

Self-reported information: the user's current main health problem or symptom.

Medical history information: such as past medical history, family medical history, chronic diseases, allergy history, etc.

Sign information: such as fever, rash, weight change, etc.

Lifestyle information: such as diet, sleep patterns, exercise, smoking and drinking habits, etc.

Auxiliary examination information: such as examination reports, imaging data, etc.

Several examples of medical consultation dialogues are given below.

### Example 1

User 1: “I'm 60 years old, and I've had persistent chest pain and shortness of breath recently, feeling a bit unwell.”

Intelligent medical consultation system: “Do you have a history of high blood pressure or heart disease? What is the onset time and duration of the chest pain?”

### Example 2

User 2: “I've had a sore throat and have been coughing for almost a week, with a slight fever at night.”

Intelligent medical consultation system: “Have you recently been in contact with anyone who has a cold or flu? Do you have a history of flu vaccination?”

User 2: “Yes, I have. My colleague has a cold, and I haven't been vaccinated.”

In an embodiment, when the target user conducts a consultation through the intelligent medical consultation dialogue interface, text, voice, or image information input by the target user is acquired.

S120, extracting disease feature information from the consultation dialogue.

Disease feature information refers to important information related to the target user's health status that is extracted from the medical consultation dialogue.

Disease feature information includes one or more of symptom information, sign information, and risk factor information.

Symptom information refers to the self-perceived, subjective physical discomfort or abnormal phenomena described by the user in the medical consultation dialogue.

As an example, symptom information may be pain, discomfort, difficulty breathing, fatigue, etc.

Sign information refers to physical signs or abnormal manifestations that can be objectively confirmed by medical examination or clinical observation.

As an example, sign information may be fever, elevated blood pressure, skin changes, sudden weight loss, etc.

Risk factor information refers to factors related to the probability of certain diseases occurring, which may increase or decrease an individual's risk of developing certain diseases.

As an example, risk factor information may be family medical history, smoking history, high blood pressure, obesity, etc.

It should be noted that, unlike traditional methods that extract feature information based on entity extraction, the solution of the present disclosure adopts a batch comparison approach, combined with a Natural Language Processing (NLP) model for task identification. This method does not rely on a fixed list of entities but automatically identifies various disease feature information described in the consultation dialogue by comparatively analyzing a large amount of case data. Specifically, the system first performs semantic analysis on the user's consultation dialogue to identify possible symptoms, signs, and risk factors, and evaluates the accuracy and relevance of this information through batch comparison, thereby extracting the disease features with the most diagnostic value.

In an embodiment, text preprocessing is performed on the user's input consultation dialogue, the text preprocessing including noise removal, word segmentation, and annotation. The consultation dialogue text is input into a natural language processing model, which can capture its contextual information and semantics. All input dialogue information is subjected to batch comparison, meaning multiple candidate disease features are simultaneously compared with the user's input dialogue to evaluate their degree of match. Semantic similarity calculation is used to measure the relevance between the user's description and each disease feature in the medical decision-making database. Based on the comparison results, the most likely disease features (such as symptoms, signs, or risk factors) are identified. The identified disease features are ranked to evaluate their importance in the entire consultation, prioritizing the extraction of the most relevant feature information. Through a task identification module, disease feature information with diagnostic value is further analyzed and extracted.

S130, querying a medical decision-making database based on the disease feature information to obtain a suspected disease set.

The medical decision-making database is a database specifically used for storing, managing, and processing data related to medical decisions, containing a large amount of medical knowledge, diagnostic criteria, relationships between disease features and symptoms, treatment plans, and disease risk assessment information.

As an example, the medical decision-making database is a pre-built database based on a large amount of clinical data, medical literature, expert experience, disease diagnosis formulas, and other content.

The medical decision-making database contains content such as disease feature information, disease diagnosis formulas, and disease treatment plans.

It can be understood that the suspected disease set includes one ore more types of suspected diseases.

In an embodiment, when the disease feature information includes a plurality of types from among symptom information, sign information, or risk factor information, step S130 may include:

S131, querying the medical decision-making database for suspected diseases corresponding to one or more types of disease feature information to obtain a plurality of suspected disease subsets, where the plurality of suspected disease subsets corresponds to the one or more type of disease feature information.

S132, comprehensively analyzing the plurality of suspected disease subsets to obtain the suspected disease set.

As an example, the suspected diseases that appear simultaneously in multiple suspected disease subsets are screened out to form the suspected disease set. That is, the union of the multiple suspected disease subsets is taken to obtain the suspected disease set.

In this embodiment, by simultaneously and comprehensively considering symptoms, signs, and risk factors, the accuracy and efficiency of screening for suspected diseases are improved, avoiding redundant judgment processes, and ultimately providing more precise and efficient support for disease diagnosis.

S140, calculating a degree of suspicion corresponding to the suspected disease in the suspected disease set.

In an embodiment, the Bayesian algorithm is used to calculate the probability value of having the disease for the suspected diseases in the suspected disease set, and this probability value is determined as the degree of suspicion corresponding to the suspected disease.

The Bayesian algorithm is a method based on probabilistic reasoning that can combine prior experience and current disease feature information to quickly and effectively perform a probability assessment of suspected diseases. The advantage of this method is that its calculation process is simple and intuitive, easy to implement, and can handle various uncertainties, providing a ranking of disease possibilities.

In an embodiment, a preset deep learning model is used to analyze the disease feature information corresponding to the suspected diseases to obtain the degree of suspicion corresponding to the suspected diseases. The preset deep learning model is trained based on samples of a plurality of disease feature information and the suspected disease labels corresponding to the plurality of disease feature information.

The preset deep learning model (such as a neural network) may self-learn the complex relationships between disease features and diseases through large-scale training data. The advantage of this method is that it can automatically extract disease feature information, handle non-linear and complex interactions between features, and has higher flexibility and accuracy, making it particularly suitable for complex and diverse disease determination scenarios.

S150, screening for a target suspected disease from the suspected disease set based on the degree of suspicion.

In an embodiment, the suspected diseases in the suspected disease set can be sorted based on the degree of suspicion, arranged from high to low. The disease with the highest degree of suspicion or the top n diseases in the sequence are then identified as the target suspected diseases.

For example, assuming the degree of suspicion ranges from 0 to 1, and the suspected disease set includes Disease A (0.9), Disease B (0.6), Disease C (0.3), and Disease D (0.2), then Disease A and Disease B would be identified as the target suspected diseases.

In an embodiment, suspected diseases from the set that reach a preset threshold for the degree of suspicion may be screened out, and these screened diseases are identified as the target suspected diseases.

For example, if a suspicion threshold of 0.7 is set, only diseases with a degree of suspicion higher than this threshold are screened out as target suspected diseases, while other diseases with a lower degree of suspicion are excluded. Applying this method to the previous example, only Disease A would be identified as the target suspected disease.

S160, determining whether an unconfirmed disease factor exists in the suspected disease based on the disease feature information.

Disease factor refers to key clinical features or conditions for diagnosing a disease, which may include key symptoms, signs, or risk factors required to confirm a disease. In an embodiment, the disease factor may also refer to a diagnostic determinant derived from disease feature information.

Diagnostic formulas for various diseases are preset in the medical decision-making database in the present disclosure presets, each diagnostic formula contains multiple disease factors related to that disease, and these factors are combined to form specific diagnostic criteria.

For example, the diagnostic formula for a certain disease might be set as:

At least 3 out of 4 preset symptoms are met.

At least 1 out of 3 preset signs is met.

When querying based on the target user's disease feature information, the system checks whether each disease factor in the diagnostic formula of the target suspected disease has been fully confirmed. Only when all relevant disease factors are confirmed to be present and meet the preset conditions will the target suspected disease be determined as the final diagnosis. If some disease factors are not yet confirmed, the system will propose additional inquiries or conduct further analysis as needed until all factors are fully verified.

S170, in response to determining that one or more unconfirmed disease factors exist in the target suspected disease, initiating a question generation task based on the unconfirmed disease factor, performing the question generation task using a large language model to obtain a question targeting the unconfirmed disease factor, and outputting the question through the intelligent medical consultation dialogue interface.

For unconfirmed disease factors, the intelligent consultation system will generate questions related to these factors using a large language model (such as GPT).

For example, if a disease factor for a certain disease is “hypertension” or “family history of illness,” the system might generate questions like the following:

“Have you measured your blood pressure recently? What was your blood pressure reading?”

“Do any of your immediate family members have a history of hypertension?”

These questions will be output to the user through the intelligent consultation dialogue interface to further inquire and supplement necessary information.

S180, in response to determining that no unconfirmed disease factor exists in the target suspected disease, determining the target suspected disease as a preliminary diagnosis result, and outputting the preliminary diagnosis result through the intelligent medical consultation dialogue interface.

Confirming that all disease factors (symptoms, signs, risk factors, etc.) of the target suspected disease have been collected and verified means that the disease now has a basis for a preliminary diagnosis. Specifically, the system will automatically evaluate the collected disease factors and match them against the diagnostic criteria of the relevant disease. If the target suspected disease meets the conditions of the diagnostic formula, the system will output a preliminary diagnosis result.

For example, if a user presents with symptoms like “fever+cough+fatigue,” the system might make a preliminary diagnosis of “upper respiratory tract infection” or “influenza.”

These preliminary diagnosis results will be communicated to the patient through the intelligent consultation dialogue interface, informing them of the preliminary diagnosis and possibly providing subsequent steps or treatment advice.

The intelligent medical consultation dialogue method provided by the present disclosure combines the natural language processing and the medical decision-making database to achieve an efficient and precise diagnostic process. Through the method, the user's symptoms, signs, and risk factors are automatically extracted, the medical decision-making database is queried to obtain a suspected disease set, and the degree of suspicion is calculated to screen for the target suspected disease. If there are one or more unconfirmed disease factors, a question is generated through a large language model to further query the user for supplementary information. Compared to traditional methods, this solution may improve consultation efficiency, diagnostic accuracy, and flexibility, while providing a transparent and controllable diagnostic process, reducing manual intervention and lowering diagnostic risks.

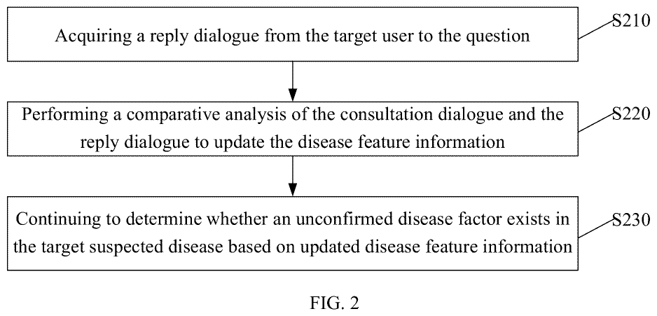

FIG. 2 is a schematic flowchart of an intelligent medical consultation dialogue method according to an embodiment of the present disclosure.

It should be understood that, in addition to steps S110 to S180, the method may also include the following steps.

S210, acquiring a reply dialogue from the target user to the question.

Through the intelligent consultation dialogue interface, relevant questions regarding unconfirmed disease factors are posed to the target user. The user provides feedback in the form of text, voice, or images. The system collects and stores the user's reply dialogue as a basis for further analysis.

S220, performing a comparative analysis of the consultation dialogue and the reply dialogue to update the disease feature information.

Using natural language processing (NLP) technology, a comparative analysis is performed on the consultation dialogue and the reply dialogue provided by the user. By identifying and extracting the user's reply information, the original disease feature information is updated.

For example, if the user adds new symptoms, signs, or risk factors, the system integrates this information with the existing disease feature information to ensure all relevant factors are confirmed and supplemented.

S230, continuing to determine whether an unconfirmed disease factor exists in the target suspected disease based on updated disease feature information.

Based on the updated disease feature information, the system re-evaluates the diagnostic formula for the target suspected diseases. If all relevant disease factors have been confirmed and meet the preset conditions, the system may further confirm the target suspected disease as the preliminary diagnosis. If there are still one or more unconfirmed disease factors, the system will again generate relevant questions and continue to ask the user in order to further collect and confirm information in the next round of consultation.

In this embodiment, disease feature information can be dynamically updated, improving the precision and flexibility of the consultation. Each user reply can potentially affect the confirmation status of disease factors, thereby precisely adjusting the judgment of suspected diseases. This closed-loop interactive approach ensures that information is continuously refined throughout the consultation process, which helps to improve the accuracy of the final diagnosis.

In an embodiment, the Bayesian algorithm is used to calculate the probability value of having the disease for each suspected disease in the suspected disease set, and this probability value is determined as the degree of suspicion corresponding to the suspected disease. The principle is as follows.

The basic form of Bayes' theorem is:

$$P ( D \vert E ) = \frac{P ( E \vert D ) \cdot P ( D )}{P ( E )} ; \qquad ( 1 )$$

Here, P(D|E) represents the probability that a patient has disease D given that evidence E (such as symptoms, signs, or risk factors) has been observed.

P(E|D) is the probability of observing evidence E given that the patient indeed has disease D, also known as the likelihood of that evidence.

P(D) is the prior probability that the patient has disease D, i.e., the probability of the patient having the disease without considering any symptoms or other diagnostic information.

P(E) is the total probability of observing evidence EE under all circumstances, by the law of total probability:

$$P ( E ) = P ( E \vert D ) \cdot P ( D ) + P ( E \vert - D ) \cdot P ( - D ) ; \qquad ( 2 )$$

Here, P(E|¬D) is the probability of observing evidence E given that the patient does not have disease D;

P(¬D) is the prior probability of not having disease D, which equals 1−P(D).

This embodiment incorporates multiple independent factors S (symptoms), Sg (signs), and Rf (risk factors), so Bayes' theorem can be extended to:

$$P ( D \vert S , Sg , Rf ) = \frac{P ( S , Sg , Rf \vert D ) \cdot P ( D )}{P ( S , Sg , Rf )} ; \qquad ( 3 )$$

P(S,Sg,Rf) is the total probability of observing symptom S, sign Sg, and risk factor Rf under all circumstances.

P(S,Sg,Rf|D) is the probability of observing symptom S, sign Sg, and risk factor Rf when the user has disease D.

P(D) is the prior probability that the user has disease D, i.e., the probability of the user having the disease without considering any symptoms or other diagnostic information.

P(D|S,Sg,Rf) is the probability that the user has disease D, given the observation of symptom S, sign Sg, and risk factor Rf.

Here, P(S,Sg,Rf), P(S,Sg,Rf|D), and P(D) in this formula are all prior probabilities. From these, it is possible to calculate the probability of the target user having each suspected disease in the suspected disease set when they present with symptom S, sign Sg, and risk factor Rf. This probability is the degree of suspicion corresponding to that suspected disease.

The advantage of this method is that its calculation process is simple and intuitive, easy to implement, and capable of handling various uncertainties, providing a ranked list of disease possibilities.

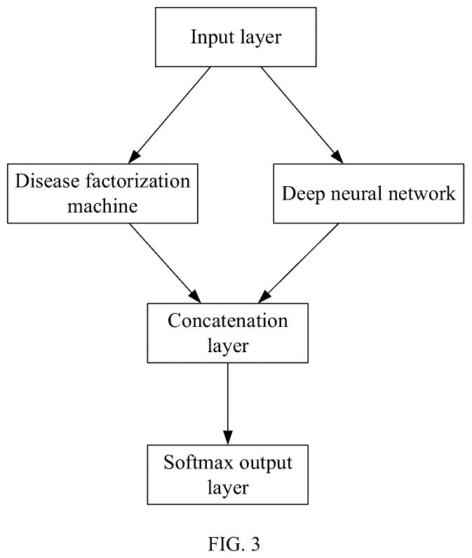

FIG. 3 is a schematic diagram showing model structure of a disease analysis model according to an embodiment of the present disclosure.

It can be understood that the disease analysis model is a hybrid model built based on a disease factorization machine and a deep neural network.

As shown in FIG. 3, the disease analysis model may include an input layer, a disease factorization machine, a deep neural network, a concatenation layer, and an output layer.

The input layer is the interface through which the model receives external data, used to introduce disease-related features.

For example, the input of the disease analysis model includes three types of medical features: symptom features, sign features, and risk factor features. These features serve as the basic input for the model for subsequent feature interaction and disease prediction.

The Disease Factorization Machine (DFM) layer is a module that handles feature interactions, focusing on modeling second-order interactions between features.

For example, the DFM constructed in this embodiment of the disclosure is as follows:

$$DFM ( S , Sg , Rf ) = {w}_{0} + {\sum }_{i = 1}^{{n}_{s}} {w}_{{s}_{i}} {s}_{i} + {\sum }_{i = 1}^{{n}_{sg}} {w}_{{sg}_{i}} {sg}_{i} + {\sum }_{i = 1}^{{n}_{rf}} {w}_{{rf}_{i}} {rf}_{i} + {\sum }_{i = 1}^{{n}_{i}} {\sum }_{j = i + 1}^{{n}_{s}} 〈 {v}_{{s}_{i}} , {v}_{{s}_{j}} 〉 {s}_{i} {s}_{j} + {\sum }_{i = 1}^{{n}_{sg}} {\sum }_{j = i + 1}^{{n}_{sg}} 〈 {v}_{{sg}_{i}} , {v}_{{sg}_{j}} 〉 {sg}_{i} {sg}_{j} + {\sum }_{i = 1}^{{n}_{rf}} {\sum }_{j = i + 1}^{{n}_{rf}} 〈 {v}_{{rf}_{i}} , {v}_{{rf}_{j}} 〉 {rf}_{i} {rf}_{j} ; \qquad ( 4 )$$

Here, sᵢ is the i-th element in the symptom feature vector. sgᵢ is the i-th element in the sign feature vector; rfᵢ is the i-th element in the risk factor vector.

w₀ is the bias or intercept term; wₛ_{ᵢ} is the weight coefficient related to the symptom feature sᵢ; w_{sg}_{ᵢ} is the weight coefficient related to the sign feature sgᵢ; w_{rf}_{ᵢ} is the weight coefficient related to the risk factor rfᵢ.

vₛᵢ is the weight matrix for the cross-terms between symptom features; v_{sg}_{ᵢ} is the weight matrix for the cross-terms between sign features; v_{rf}_{ᵢ} is the weight matrix for the cross-terms between risk factor features.

<⋅,⋅>: the inner product operator, used to calculate the dot product of two vectors.

By independently modeling the second-order interactions of symptoms, signs, and risk factors respectively, the DFM can capture the complex associations between these medical features. Its goal is to improve the model's sensitivity and accuracy regarding disease-related features, especially in its performance on second-order feature interactions.

The Deep Neural Network (DNN) layer is used for the extraction of high-order non-linear features.

The DNN layer may directly perform high-order feature extraction on the input features, learning deep non-linear interaction patterns between features (e.g., the potential relationship between chest pain and smoking history with cardiovascular disease). This supplements the low-order information provided by the DFM. By capturing complex disease associations through high-order patterns, it enhances the model's predictive capabilities.

For example, the DNN constructed in this embodiment is as follows:

$$\begin{matrix}x = concat ( S , {S}_{g} , {R}_{f} ) \\ {h}^{( l + 1 )} = σ ( {W}^{( l )} {h}^{( l )} + {b}^{( l )} )\end{matrix} ; \qquad ( 5 )$$

x=concat(S, S_{g}, R_{f}) indicates that the three input features (symptoms S, signs S_{g}, and risk factors R_{f}) are concatenated to form a comprehensive feature vector x.

h^{(l+1)}=σ(W^{(l)}h^{(l)}+b^{(l)}) indicates that in the DNN, the output w^{(l)} of layer l is applied through an activation function σ to the product of the weight matrix w^{(l)} and the bias vector b^{(l)}, yielding the output h^{(l+1)} of the next layer (layer l+1). This process is the core step of feature extraction and learning within the DNN.

For example, the activation function a can be the Rectified Linear Unit (ReLU).

For example, the number of layers L=64.

The concatenation layer (Concat) is an integration module used to combine the output information from different layers.

In the concatenation layer, the second-order interaction information output by the DFM layer is integrated with the high-order non-linear features extracted by the DNN layer to form a comprehensive feature vector. This vector contains both low-order interaction information and high-order complex features, providing comprehensive data support for subsequent disease prediction.

The Softmax output layer is a normalized output module used to generate a probability distribution for the target disease categories.

The Softmax output layer receives the comprehensive feature vector from the concatenation layer and transforms this comprehensive feature vector into a probability distribution for the target disease categories.

The disease analysis model provided in this embodiment introduces disease features such as symptoms, signs, and risk factors through an input layer, captures low-order feature interactions using a Disease Factorization Machine, extracts high-order complex feature interactions with a deep neural network, integrates low-order and high-order information in a concatenation layer, and finally outputs the probability distribution of the target disease through a Softmax layer. This architecture considers multi-level feature modeling and information fusion, enabling it to efficiently capture complex correlations among various disease features, thereby improving the accuracy and interpretability of disease prediction.

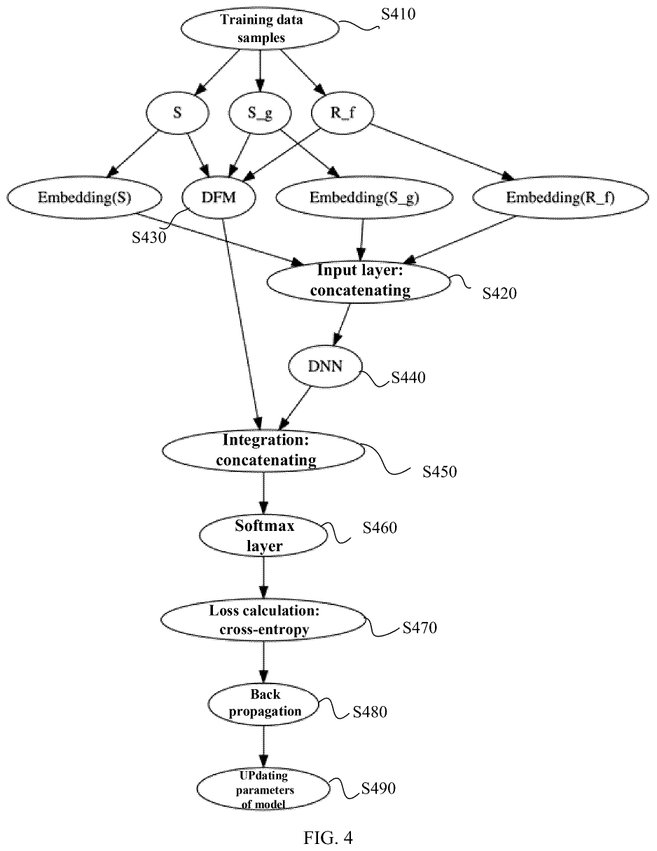

FIG. 4 is a schematic flowchart of a training method for a disease analysis model according to an embodiment of the present disclosure.

It should be understood that this disease analysis model is used to analyze disease feature information corresponding to a suspected disease to obtain a degree of suspicion for the suspected disease.

As shown in FIG. 4, the training method for this model may include the following steps.

S410, acquiring training data samples.

In an embodiment, the method for acquiring training data samples may include the following steps.

S411, constructing a disease library.

For example, a disease library may be constructed based on the International Classification of Diseases, 10th Revision (ICD-10) codes.

The disease library includes the corresponding symptoms, signs, and risk factors associated with each disease.

S412, constructing feature vectors.

A pre-trained embedding model is used to convert the text descriptions of each symptom, sign, and risk factor into feature vectors.

For example, the pre-trained embedding model may be Word2Vec or Bidirectional Encoder Representations from Transformers (BERT).

S413, constructing training data samples.

Each sample contains a disease label and corresponding multiple symptom feature vectors, sign feature vectors, and risk factor feature vectors.

Table 1 shows the feature data and feature vector data for diabetes.

| | | Symptom | | | | |
|---|---|---|---|---|---|---|
| | | Feature | | Sign Feature | Risk | Risk Factor |
| Disease | Symptom | Vector | Sign | Vector | Factor | Feature Vector |
| Diabetes | Polydipsia | [0.23, −0.12, | Hyperglycemia | [0.30, −0.15, | Age >45 | [0.15, 0.25, . . . , |
| | | . . . , 0.45] | | . . . , 0.50] | | 0.35] |
| | Polyuria | [0.18, 0.34, | Ketosis | [0.28, 0.14, | Family | [0.20, 0.10, . . . , |
| | | . . . , 0.39] | | . . . , 0.48] | history | 0.40] |
| | | | | | of | |
| | | | | | diabetes | |
| | Unexplained | [0.25, 0.04, | Hypertension | [0.22, −0.05, | Obesity | [0.22, 0.05, . . . , |
| | weight | . . . , 0.55] | | . . . , 0.44] | | 0.38] |
| | loss | | | | | |
| | Fatigue | [0.16, −0.24, | | | Lack of | [0.18, 0.22, . . . , |
| | | . . . , 0.33] | | | exercise | 0.36] |
| | Blurred | [0.19, 0.21, | | | Unhealthy | [0.21, 0.17, . . . , |
| | vision | . . . , 0.42] | | | diet | 0.41] |

Table 1 shows the disease feature vectors for diabetes. When inputting into the model, the feature vectors in the table can be concatenated.

For example, all symptom feature vectors are concatenated into a long vector, resulting in [0.23, −0.12, . . . , 0.45, 0.18, 0.34, . . . , 0.39, 0.25, 0.04, . . . , 0.55, 0.16, −0.24, . . . , 0.33, 0.19, 0.21, . . . , 0.42].

All sign feature vectors are concatenated into a long vector, resulting in [0.30, −0.15, . . . , 0.50, 0.28, 0.14, . . . , 0.48, 0.22, −0.05, . . . , 0.44].

All risk factor feature vectors are concatenated into a long vector, resulting in [0.15, 0.25, . . . , 041].

S420, The input layer concatenating S, S_{g}, R_{f} into a vector x.

The input layer receives feature data from multiple sources, including the embedding representations of symptoms, signs, and risk factors. After these features are standardized, encoded, or embedded, they are concatenated into a unified vector x according to a preset dimension, serving as the input for subsequent layers.

S430, calculating second-order feature interactions using DFM layer.

In the DFM layer, the second-order interaction relationships between input features may be calculated using the factorization technique shown in formula (4).

S440, performing forward propagation through a multi-layer perceptron based on vector x using DNN layer.

In the DNN layer, with vector x as input, non-linear feature extraction is performed through a multi-layer perceptron as shown in formula (5).

S450, concatenating the outputs of DFM and DNN.

In the concatenation layer, the second-order feature interaction information from the DFM layer and the high-order feature extraction results from the DNN layer are integrated into a comprehensive feature vector.

S460, generating a probability distribution using softmax layer.

The comprehensive feature vector is input to the Softmax output layer, which maps it to a probability distribution of the target disease categories.

S470, calculating a cross-entropy loss based on the predicted probabilities and true labels.

For example, the formula for the Loss function is as follows:

$$ℒ = - {\sum }_{i = 1}^{N} log ( \hat{P} ( {D}_{i} \vert {S}_{i} , {S}_{{g}_{i}} , {R}_{{f}_{i}} ) ) ; \qquad ( 6 )$$

S480, performing back propagation through the loss.

Using the calculated cross-entropy loss, the disease analysis model uses a back propagation algorithm to compute gradients, updating the weights and bias parameters of each layer sequentially from the output layer to the input layer.

S490, updating parameters of model.

The parameters of the disease analysis model are updated, finally obtaining the trained disease analysis model.

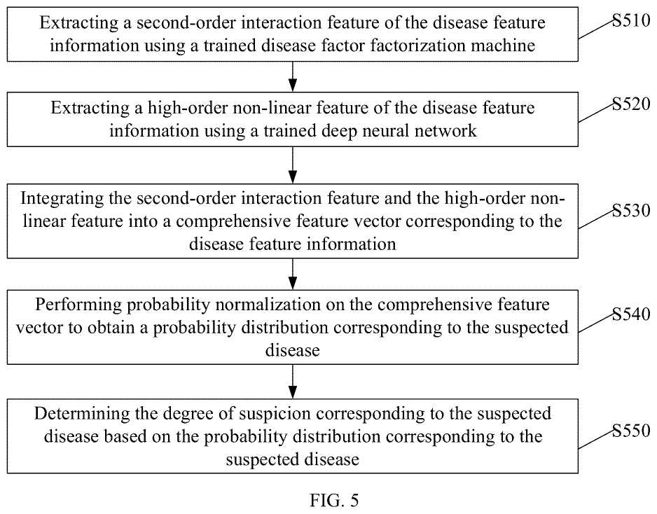

FIG. 5 is a schematic flowchart of a prediction method for a disease analysis model according to an embodiment of the present disclosure.

It should be understood that using the pre-trained disease analysis model to analyze the disease feature information corresponding to a suspected disease and obtain the degree of suspicion for the suspected disease may include the following steps.

S510, extracting a second-order interaction feature of the disease feature information using a trained disease factor factorization machine.

S520, extracting a high-order non-linear feature of the disease feature information using a trained deep neural network.

S530, integrating the second-order interaction feature and the high-order non-linear feature into a comprehensive feature vector corresponding to the disease feature information.

S540, performing probability normalization on the comprehensive feature vector to obtain a probability distribution corresponding to the suspected disease.

S550, determining the degree of suspicion corresponding to the suspected disease based on the probability distribution corresponding to the suspected disease.

That is, by using the trained disease analysis model mentioned above to analyze the disease feature information for each suspected disease, a probability distribution for each suspected disease is output, and the disease with the highest probability is selected as the final prediction result.

Through this method, disease feature information may be automatically extracted, and interactions between non-linear and complex features may also be processed, offering higher flexibility and accuracy, thus this method is particularly suitable for complex and diverse disease diagnosis scenarios.

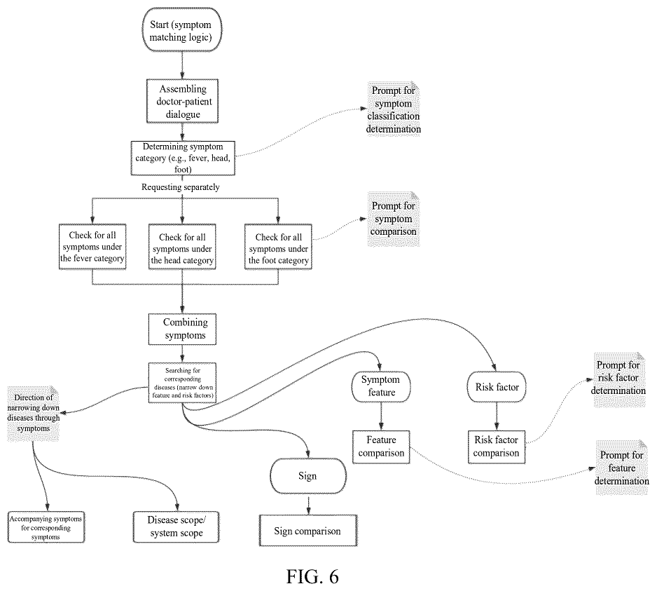

FIG. 6 is a schematic flowchart of an intelligent medical consultation dialogue method according to an embodiment of the present disclosure.

As shown in FIG. 6, the method may include the following processes.

It should be understood that the doctor agent mentioned below is an example of the intelligent consultation system in the present disclosure, which executes the intelligent consultation dialogue method provided in the disclosed embodiments.

For example, the doctor agent may be a virtual medical assistant designed based on the method of the present disclosure, whose core function is to conduct consultation dialogues and disease diagnosis with users through natural language processing, medical decision database, and deep learning technology.

Start (symptom matching): the patient describes their symptoms to the doctor agent, and the doctor agent begins matching these symptoms to determine possible diseases.

Assemble doctor-patient dialogue content: the doctor agent records the patient's symptom descriptions for further analysis.

Symptom-based diagnostic classification: based on the patient's symptoms, the doctor agent may provide some possible disease diagnoses.

Determine symptom category (e.g., fever, head, foot): the doctor agent classifies the patient's symptoms into different categories, such as fever, head symptoms, or foot symptoms.

Request separately: the doctor agent may ask the patient for more information about specific symptoms for a more accurate diagnosis.

Check for all symptoms under the fever category: the doctor agent checks the patient's symptoms under the fever category to determine if relevant symptoms are present.

Check for all symptoms under the head category: the doctor agent checks the patient's symptoms under the head category to determine if relevant symptoms are present.

Check for all symptoms under the foot category: the doctor agent checks the patient's symptoms under the foot category to determine if relevant symptoms are present.

Combine symptoms: the doctor agent combines the patient's symptoms to determine possible diseases.

Narrow down disease possibilities with symptoms: the doctor agent narrows down the range of possible diseases based on the patient's symptoms.

Accompanying symptoms for corresponding symptoms: the doctor agent checks if the patient has other symptoms related to the main symptoms.

Disease scope/system scope: the doctor agent determines the possible disease scope or system involved in the patient's symptoms.

Feature comparison: the doctor agent compares the patient's symptom features with known diseases to determine possible diseases.

Risk factor comparison: the doctor agent compares the patient's risk factors with known diseases to determine possible diseases.

Symptom comparison: The doctor agent compares the patient's symptoms with known diseases to determine possible diseases.

End: the doctor agent determines the possible diseases based on the patient's symptoms, risk factors, and symptom features, and provides the diagnosis result to the patient.

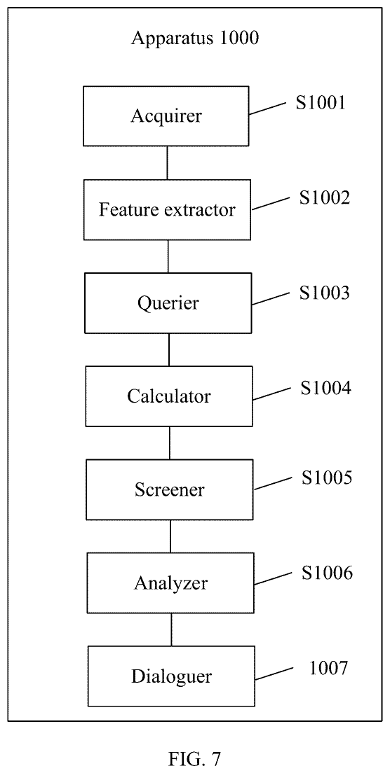

FIG. 7 is a schematic structural diagram of an intelligent medical consultation dialogue apparatus according to an embodiment of the present disclosure.

As shown in FIG. 7, the intelligent medical consultation dialogue apparatus 1000 may include the following parts:
an acquirer 1001, configured to acquire a medical consultation dialogue from a target user through an intelligent medical consultation dialogue interface;
a feature extractor 1002, configured to extract disease feature information from the consultation dialogue, where the disease feature information includes one or more of symptom information, sign information, and risk factor information;
a querier 1003, configured to query a medical decision-making database based on the disease feature information to obtain a suspected disease set, where the suspected disease set contains one or more suspected diseases;
a calculator 1004, configured to calculate a degree of suspicion corresponding to the suspected disease in the suspected disease set;
a screener 1005, configured to screen for a target suspected disease from the suspected disease set based on the degree of suspicion;
an analyzer 1006, configured to determine whether an unconfirmed disease factor exists in the target suspected disease based on the disease feature information; and
a dialoguer 1007, configured to, in response to determining that one or more unconfirmed disease factors exist in target the suspected disease, initiate a question generation task based on the unconfirmed disease factor, perform the question generation task using a large language model to obtain a question targeting the unconfirmed disease factor, and output the question through the intelligent medical consultation dialogue interface.

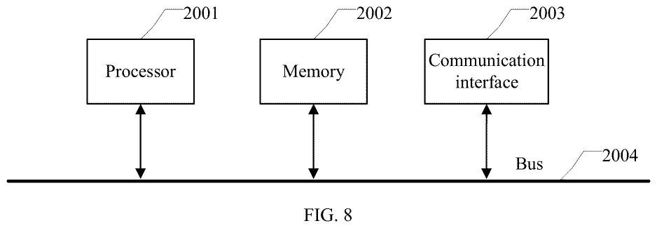

FIG. 8 is a schematic structural diagram of a computer device according to an embodiment of the present disclosure.

The computer device may include a processor 2001 and a memory 2002 storing computer programs or instructions.

In an embodiment, the aforementioned processor 2001 may include a Central Processing Unit (CPU), or an Application Specific Integrated Circuit (ASIC), or may be configured to implement one or more integrated circuits of the embodiments of the present disclosure.

The memory 2002 may include mass storage for data or instructions. For example, but not limited to, the memory 2002 may include a Hard Disk Drive (HDD), a floppy disk drive, flash memory, a compact disk, a magneto-optical disk, a magnetic tape, a Universal Serial Bus (USB) drive, or a combination of two or more of these. In an example, the memory 2002 may include removable or non-removable (or fixed) media, or the memory 2002 may be non-transitory solid-state memory. The memory 2002 may be internal or external to the integrated gateway disaster recovery device.

In an embodiment, the memory 2002 may be read only memory (ROM). In an example, the ROM may be a mask-programmed ROM, a programmable ROM (PROM), an erasable PROM (EPROM), an electrically erasable PROM (EEPROM), an electrically alterable ROM (EAROM), or flash memory, or a combination of two or more of these.

The memory 2002 may include read-only memory (ROM), random access memory (RAM), disk storage media devices, optical storage media devices, flash memory devices, or electrical, optical, or other physical/tangible memory storage devices. Therefore, in general, the memory 2002 includes one or more tangible (non-transitory) computer-readable storage media (e.g., memory devices) encoded with software including computer-executable instructions, and when the software is executed (e.g., by one or more processors), it is operable to perform the operations described with reference to the methods according to the first aspect of the present disclosure.

The processor 2001 implements the method in the embodiment shown in FIG. 1 by reading and executing the computer program instructions stored in the memory 2002.

Additionally, in conjunction with the methods in the above embodiments, the embodiments of the present disclosure may provide a non-transitory computer storage medium to implement them. The computer storage medium stores computer program instructions; when the computer program instructions are executed by a processor, they implement any of the methods in the above embodiments.

An embodiment of the present disclosure further provides a computer program product, which includes a computer program. When the computer program is executed by a processor, it implements any of the methods in the above embodiments.

It should be clarified that the present disclosure is not limited to the specific configurations and processes described above and shown in the drawings. For the sake of brevity, detailed descriptions of known methods are omitted here. In the above embodiments, several specific steps are described and shown as examples. However, the method process of the present disclosure is not limited to the specific steps described and shown. Those skilled in the art can make various changes, modifications, and additions, or change the order between steps, after understanding the principle of the present disclosure.

The functional blocks shown in the structural block diagrams described above may be implemented as hardware, software, firmware, or a combination thereof. When implemented in hardware, it can be, for example, an electronic circuit, an application specific integrated circuit (ASIC), appropriate firmware, a plug-in, a function card, and so on. When implemented in software, the elements of the present disclosure are programs or code segments used to perform the required tasks. The program or code segments can be stored in a machine-readable medium or transmitted over a transmission medium or communication link via a data signal carried in a carrier wave. A “machine-readable medium” may include any medium capable of storing or transmitting information. Examples of machine-readable media include electronic circuits, semiconductor memory devices, read-only memory (ROM), flash memory, erasable read only memory (EROM), floppy disks, compact disc read-only memory (CD-ROM), optical disks, hard disks, fiber optic media, radio frequency (RF) links, and so on. The code segments may be downloaded via computer networks such as the Internet, intranets, etc.

It also needs to be stated that the exemplary embodiments mentioned in the present disclosure describe some methods or systems based on a series of steps or devices. However, the present disclosure is not limited to the order of the steps described above, that is, the steps can be executed in the order mentioned in the embodiments, or in a different order from the embodiments, or several steps can be executed simultaneously.

The various aspects of the present disclosure have been described above with reference to flowcharts and/or block diagrams of methods, apparatuses (systems), and computer program products according to embodiments of the present disclosure. It should be understood that each block in the flowcharts and/or block diagrams, and combinations of blocks in the flowcharts and/or block diagrams, can be implemented by computer program instructions. These computer program instructions may be provided to the processor of a general-purpose computer, a special-purpose computer, or other programmable data processing apparatus to produce a machine, such that these instructions, when executed via the processor of the computer or other programmable data processing apparatus, enable the implementation of the functions/actions specified in one or more blocks of the flowchart and/or block diagram. Such a processor may be, but is not limited to, a general-purpose processor, a special-purpose processor, a special application processor, or a field-programmable logic circuit. It can also be understood that each block in the block diagrams and/or flowcharts, and combinations of blocks in the block diagrams and/or flowcharts, can also be implemented by dedicated hardware that performs the specified functions or actions, or can be implemented by a combination of dedicated hardware and computer instructions.

The foregoing are only specific embodiments of the present disclosure. It can be clearly understood by those skilled in the art that, for the convenience and brevity of description, the specific working processes of the systems, modules, and units described above can be referred to the corresponding processes in the foregoing method embodiments, and will not be repeated here. It should be understood that the scope of protection of the present disclosure is not limited to this. Any person skilled in the art can easily conceive of various equivalent modifications or replacements within the technical scope disclosed by the present disclosure, and these modifications or replacements should be covered within the scope of protection of the present disclosure.

What is claimed is:

1. An intelligent medical consultation dialogue method, comprising:
acquiring a medical consultation dialogue from a target user through an intelligent medical consultation dialogue interface;
extracting disease feature information from the consultation dialogue, wherein the disease feature information comprises one or more of symptom information, sign information, and risk factor information;
querying a medical decision-making database based on the disease feature information to obtain a suspected disease set, wherein the suspected disease set contains one or more suspected diseases;
calculating a degree of suspicion corresponding to the suspected disease in the suspected disease set;
screening for a target suspected disease from the suspected disease set based on the degree of suspicion;
determining whether an unconfirmed disease factor exists in the suspected disease based on the disease feature information; and
in response to determining that one or more unconfirmed disease factors exist in the target suspected disease, initiating a question generation task based on the unconfirmed disease factor, performing the question generation task using a large language model to obtain a question targeting the unconfirmed disease factor, and outputting the question through the intelligent medical consultation dialogue interface.

2. The method according to claim 1, further comprising:
in response to determining that no unconfirmed disease factor exists in the target suspected disease, determining the target suspected disease as a preliminary diagnosis result, and outputting the preliminary diagnosis result through the intelligent medical consultation dialogue interface.

3. The method according to claim 1, wherein when the disease feature information comprises a plurality of the symptom information, the sign information, and the risk factor information, the querying a medical decision-making database based on the disease feature information to obtain a suspected disease set comprises:
querying the medical decision-making database for suspected diseases corresponding to one or more types of disease feature information to obtain a plurality of suspected disease subsets, wherein the plurality of suspected disease subsets corresponds to the one or more type of disease feature information; and
comprehensively analyzing the plurality of suspected disease subsets to obtain the suspected disease set.

4. The method according to claim 1, wherein the calculating a degree of suspicion corresponding to the suspected disease in the suspected disease set comprises:
calculating a disease probability value for the one or more suspected diseases in the suspected disease set using a Bayesian algorithm, and determining the disease probability value as the degree of suspicion corresponding to the suspected disease.

5. The method according to claim 1, wherein the calculating a degree of suspicion corresponding to the suspected disease in the suspected disease set comprises:
analyzing the disease feature information corresponding to the suspected disease to obtain the degree of suspicion corresponding to the suspected disease using a disease analysis model, wherein the disease analysis model is trained based on samples of a plurality of disease feature information and suspected disease labels corresponding to the plurality of disease feature information.

6. The method according to claim 5, wherein the disease analysis model is a hybrid model constructed based on a disease factor factorization machine and a deep neural network.

7. The method according to claim 6, wherein the analyzing the disease feature information corresponding to the suspected disease to obtain the degree of suspicion corresponding to the suspected disease using a disease analysis model comprises:
extracting a second-order interaction feature of the disease feature information using a trained disease factor factorization machine; and
extracting a high-order non-linear feature of the disease feature information using a trained deep neural network;
integrating the second-order interaction feature and the high-order non-linear feature into a comprehensive feature vector corresponding to the disease feature information;
performing probability normalization on the comprehensive feature vector to obtain a probability distribution corresponding to the suspected disease; and
determining the degree of suspicion corresponding to the suspected disease based on the probability distribution corresponding to the suspected disease.

8. The method according to claim 1, wherein the screening for a target suspected disease from the suspected disease set based on the degree of suspicion comprises:
screening for a suspected disease from the suspected disease set whose degree of suspicion reaches a preset threshold, and determining the screened suspected disease as the target suspected disease.

9. The method according to claim 1, further comprising:
acquiring a reply dialogue from the target user to the question;
performing a comparative analysis of the consultation dialogue and the reply dialogue to update the disease feature information; and
continuing to determine whether an unconfirmed disease factor exists in the target suspected disease based on updated disease feature information.

10. An intelligent medical consultation dialogue apparatus, comprising:
an acquirer, configured to acquire a medical consultation dialogue from a target user through an intelligent medical consultation dialogue interface;
a feature extractor, configured to extract disease feature information from the consultation dialogue, wherein the disease feature information comprises one or more of symptom information, sign information, and risk factor information;
a querier, configured to query a medical decision-making database based on the disease feature information to obtain a suspected disease set, wherein the suspected disease set contains one or more suspected diseases;
a calculator, configured to calculate a degree of suspicion corresponding to the suspected disease in the suspected disease set;
a screener, configured to screen for a target suspected disease from the suspected disease set based on the degree of suspicion;
an analyzer, configured to determine whether an unconfirmed disease factor exists in the target suspected disease based on the disease feature information; and
a dialoguer, configured to, in response to determining that one or more unconfirmed disease factors exist in target the suspected disease, initiate a question generation task based on the unconfirmed disease factor, perform the question generation task using a large language model to obtain a question targeting the unconfirmed disease factor, and output the question through the intelligent medical consultation dialogue interface.

11. A non-transitory computer-readable storage medium, having computer programs or instructions stored thereon, wherein the computer programs or instructions, when executed by a processor, cause the processor to perform acts comprising:
acquiring a medical consultation dialogue from a target user through an intelligent medical consultation dialogue interface;
extracting disease feature information from the consultation dialogue, wherein the disease feature information comprises one or more of symptom information, sign information, and risk factor information;
querying a medical decision-making database based on the disease feature information to obtain a suspected disease set, wherein the suspected disease set contains one or more suspected diseases;
calculating a degree of suspicion corresponding to the suspected disease in the suspected disease set;
screening for a target suspected disease from the suspected disease set based on the degree of suspicion;
determining whether an unconfirmed disease factor exists in the suspected disease based on the disease feature information; and
in response to determining that one or more unconfirmed disease factors exist in the target suspected disease, initiating a question generation task based on the unconfirmed disease factor, performing the question generation task using a large language model to obtain a question targeting the unconfirmed disease factor, and outputting the question through the intelligent medical consultation dialogue interface.

12. The storage medium according to claim 11, wherein the processor further performs acts comprising:
in response to determining that no unconfirmed disease factor exists in the target suspected disease, determining the target suspected disease as a preliminary diagnosis result, and outputting the preliminary diagnosis result through the intelligent medical consultation dialogue interface.

13. The storage medium according to claim 11, wherein when the disease feature information comprises a plurality of the symptom information, the sign information, and the risk factor information, the querying a medical decision-making database based on the disease feature information to obtain a suspected disease set comprises:
querying the medical decision-making database for suspected diseases corresponding to one or more types of disease feature information to obtain a plurality of suspected disease subsets, wherein the plurality of suspected disease subsets corresponds to the one or more type of disease feature information; and
comprehensively analyzing the plurality of suspected disease subsets to obtain the suspected disease set.

14. The storage medium according to claim 11, wherein the calculating a degree of suspicion corresponding to the suspected disease in the suspected disease set comprises:
calculating a disease probability value for the one or more suspected diseases in the suspected disease set using a Bayesian algorithm, and determining the disease probability value as the degree of suspicion corresponding to the suspected disease.

15. The storage medium according to claim 11, wherein the calculating a degree of suspicion corresponding to the suspected disease in the suspected disease set comprises:
analyzing the disease feature information corresponding to the suspected disease to obtain the degree of suspicion corresponding to the suspected disease using a disease analysis model, wherein the disease analysis model is trained based on samples of a plurality of disease feature information and suspected disease labels corresponding to the plurality of disease feature information.

16. The storage medium according to claim 15, wherein the disease analysis model is a hybrid model constructed based on a disease factor factorization machine and a deep neural network.

17. The storage medium according to claim 16, wherein the analyzing the disease feature information corresponding to the suspected disease to obtain the degree of suspicion corresponding to the suspected disease using a disease analysis model comprises:
extracting a second-order interaction feature of the disease feature information using a trained disease factor factorization machine; and
extracting a high-order non-linear feature of the disease feature information using a trained deep neural network;
integrating the second-order interaction feature and the high-order non-linear feature into a comprehensive feature vector corresponding to the disease feature information;
performing probability normalization on the comprehensive feature vector to obtain a probability distribution corresponding to the suspected disease; and
determining the degree of suspicion corresponding to the suspected disease based on the probability distribution corresponding to the suspected disease.

18. The storage medium according to claim 11, wherein the screening for a target suspected disease from the suspected disease set based on the degree of suspicion comprises:
screening for a suspected disease from the suspected disease set whose degree of suspicion reaches a preset threshold, and determining the screened suspected disease as the target suspected disease.

19. The storage medium according to claim 11, wherein the processor further performs acts comprising:
acquiring a reply dialogue from the target user to the question;
performing a comparative analysis of the consultation dialogue and the reply dialogue to update the disease feature information; and
continuing to determine whether an unconfirmed disease factor exists in the target suspected disease based on updated disease feature information.
